(19) United States
(12) Patent Application Publication
(10) Pub. No.: US 2004/0214178 A1
(43) Pub. Date: Oct. 28, 2004

(54) Reporter-selectable Hepatitis C virus replicon

(75) Inventors: Rohit Duggal, San Diego, CA (US); Amy Karen Patick, Escondido, CA (US); Jie Zhang, Carlsbad, CA (US); Weidong Zhao, San Diego, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 10/422,323

(22) Filed: Apr. 24, 2003

(51) Int. Cl. C12Q 1/70; C12Q 1/68; C07H 21/04; C12P 1/00

(52) U.S. Cl. 435/6; 536/23.72; 435/41

(57) ABSTRACT

The invention relates to a reporter-selectable hepatitis C virus (HCV) replicon, and use of the replicon to generate stable, human hepatoma cell lines. The replicon and cell lines are useful in the compound screening process in HCV drug discovery.

### FIELD OF THE INVENTION

The invention relates to a reporter-selectable Hepatitis C virus (HCV) replicon and use of the replicon to generate stable, human hepatoma cell lines. The replicon and cell lines are useful in the compound screening process in HCV drug discovery.

### BACKGROUND OF THE INVENTION

Hepatitis C virus is a member of the hepacivirus genus in the family Flaviviridae. It is the major causative agent of non-A, non-B viral hepatitis and is the major cause of transfusion-associated hepatitis and accounts for a significant proportion of hepatitis cases worldwide. Although acute HCV infection is often asymptomatic, nearly 80% of cases resolve to chronic hepatitis. The persistent property of the HCV infection has been explained by its ability to escape from the host immune surveillance through hypermutability of the exposed regions in the envelope protein E2 (Weiner et al., Virology 180:842-848 (1991); Weiner et al. Proc. Natl. Acad. Sci. USA 89:3468-3472 (1992)). About 60% of patients develop liver disease with various clinical outcomes ranging from an asymptomatic carrier state to chronic active hepatitis and liver cirrhosis (occurring in about 20% of patients), which is strongly associated with the development of hepatocellular carcinoma (occurring in about 1-5% of patients). See Cuthbert, Clin. Microbiol. Rev. 7:505-532 (1994); World Health Organization, Lancet 351:1415 (1998). The World Health Organization estimates that 170 million people are chronically infected with HCV, with an estimate of 4 million of these living in the United States.

HCV is an enveloped ribonucleic acid (RNA) virus containing a single-stranded positive-sense RNA genome approximately 9.5 kb in length (Choo et al., Science 244:359-362 (1989)). The RNA genome contains a 5′-nontranslated region (5′ NTR) of 341 nucleotides (Brown et al., Nucl. Acids Res. 20:5041-5045 (1992); Bukh et al., Proc. Natl. Acad. Sci. USA 89:4942-4946 (1992)), a large open reading frame (ORF) encoding a single polypeptide of 3,010 to 3,040 amino acids (Choo et al. (1989), supra), and a 3′-nontranslated region (3′ NTR) of variable length of about 230 nucleotides (Kolykhalov et al., J. Virol. 70:3363-3371 (1996); Tanaka et al., J. Virol. 70:3307-3312 (1996)). By analogy to other plus-strand RNA viruses, the 3′ nontranslated region is assumed to play an important role in viral RNA synthesis. HCV is similar in amino acid sequence and genome organization to flaviviruses and pestiviruses (Miller et al., Proc. Natl. Acad. Sci. USA 87:2057-2061 (1990)), and therefore HCV has been classified as a third genus of the family Flaviviridae (Francki et al., Arch. Virol. 2:223-233 (1991)).

Studies of HCV replication and the search for specific anti-HCV agents have been hampered by the lack of an efficient tissue culture system for HCV propagation, the absence of a suitable small-animal model for HCV infection, the low level of viral replication, and the considerable genetic heterogeneity associated with the virus (Bartenschlager, Antivir. Chem. Chemother. 8:281-301 (1997); Simmonds et al., J. Gen. Virol. 74:2391-2399 (1993)). The current understanding of the structures and functions of the HCV genome and encoded proteins is primarily derived from in vitro studies using various recombinant systems (Bartenschlager (1997), supra).

The 5′ NTR is one of the most conserved regions of the viral genome and plays a pivotal role in the initiation of translation of the viral polyprotein (Bartenschlager (1997), supra). A single long ORF encodes a polyprotein, which is co- or post-translationally processed into structural (core, E1, and E2) and nonstructural (NS2, NS3, NS4A, NS4B, NS5A, and NS5B) viral proteins by either cellular or viral proteinases (Bartenschlager (1997), supra). The 3′ NTR consists of three distinct regions: a variable region of about 38 nucleotides following the stop codon of the polyprotein, a polyuridine tract of variable length with interspersed substitutions of cytosines, and 98 nucleotides (nt) at the very 3′ end which are highly conserved among various HCV isolates. The order of the genes within the genome is: NH₂-C-E1-E2-p7-NS2-NS3-NS4A-NS4B-NS5A-NS5B-COOH (Grakoui et al., J. Virol. 67:1385-1395 (1993)).

Processing of the structural proteins core (C), envelope protein 1 and (E1, E2), and the p7 region is mediated by host signal peptidases. In contrast, maturation of the nonstructural (NS) region is accomplished by two viral enzymes. The HCV polyprotein is first cleaved by a host signal peptidase generating the structural proteins C/E1, E1/E2, E2/p7, and p7/NS2 (Hijikata et al., Proc. Natl. Acad. Sci. USA 88:5547-5551 (1991); Lin et al., J. Virol. 68:5063-5073 (1994)). The NS2-3 proteinase, which is a metalloprotease, then cleaves at the NS2/NS3 junction. The NS3/4A proteinase complex (NS3 being a serine protease and NS4A acting as a cofactor of the NS3 protease) is then responsible for processing at all the remaining sites (Bartenschlager et al., J. Virol. 67:3835-3844 (1993); Bartenschlager (1997), supra). RNA helicase and NTPase activities have also been identified in the NS3 protein. The N-terminal one-third of the NS3 protein functions as a protease, and the remaining two-thirds of the molecule acts as the helicase/ATPase that is thought to be involved in HCV replication. NS4A is a cofactor for the NS3 protease and is followed by NS4B, for which the function is unknown. NS5A is a phosphorylated protein and its function is currently unknown. The fourth viral enzyme, NS5B, is an RNA-dependent RNA polymerase (RdRp) and a key component responsible for replication of the viral RNA genome (Lohmann et al., J. Virol. 71:8416-8428 (1997)). NS5B contains the “GDD” sequence motif, which is highly conserved among all RdRps characterized to date (Poch et al., EMBO J. 8:3867-3874 (1989)).

Replication of HCV is thought to occur in membrane-associated replication complexes. Within these, the genomic plus-strand RNA is used as a template to synthesize minus-strand RNA, which in turn can be used for the synthesis of progeny genomic plus strands. At least two viral proteins appear to be involved in this process: the NS3 protein, which carries in the carboxy terminal two-thirds a nucleoside triphosphatase/RNA helicase, and the NS5B protein, which has RdRp activity. See Hwang et al., J. Virol. 227:439-446 (1997).

While the role of NS3 in RNA replication is less clear, NS5B apparently is the key enzyme responsible for synthesis of progeny RNA strands. Using recombinant baculoviruses to express NS5B in insect cells and a synthetic nonviral RNA as a substrate, NS5B was found to possess a primer-dependent RdRp activity. It was subsequently confirmed and further characterized through the use of the HCV RNA genome as a substrate (Lohmann et al., Virology 249:108-118 (1998)). Recent studies have shown that NS5B with a C-terminal 21 amino-acid truncation expressed in Escherichia coli is also active to carry out in vitro RNA synthesis (Ferrari et al., J. Virol. 73:1649-1654 (1999); Yamashita et al., J. Biol. Chem. 273:15479-15486 (1998)).

Since persistent infection of HCV is related to chronic hepatitis and eventually to hepatocarcinogenesis, HCV replication is one of the targets to eliminate HCV reproduction and to prevent hepatocellular carcinoma. Unfortunately, present treatment approaches for HCV infection are marked by relatively poor efficacy and unfavorable side effects. Therefore, intensive research is directed to the discovery of molecules to treat this disease. New approaches include the development of prophylactic and therapeutic vaccines, the identification of interferons with improved pharmacokinetic characteristics, and the discovery of drugs designed to inhibit the function of the three key HCV proteins: protease, helicase and polymerase. Also, the HCV RNA genome itself, particularly the internal ribosome entry site (IRES), is being explored as an antiviral target using antisense molecules and catalytic ribozymes. For a review, see Wang et al., Prog. Drug Res. 55:1-32 (2000).

Particular therapies for HCV infection include α-interferon alone and the combination of α-interferon with ribavirin. These therapies have been shown to be effective in a portion of patients with chronic HCV infection (Marcellin et al., Ann. Intern. Med. 127:875-881 (1997); Zeuzem et al., Hepatology 28:245-252 (1998)). Use of antisense oligonucleotides for treatment of HCV infection has also been proposed, e.g, Anderson et al., U.S. Pat. No. 6,174,868 (2001), as well as use of free bile acids, such as ursodeoxycholic acid and chenodeoxycholic acid, or conjugated bile acids, such as tauroursodeoxycholic acid (Ozeki, U.S. Pat. No. 5,846,964 (1998)). Phosphonoformic acid esters have also been proposed to be useful in treating a number of viral infections including HCV (Helgstrand et al., U.S. Pat. No. 4,591,583 (1986)). However, vaccine development has been hampered by the high degree of immune evasion and the lack of protection against re-infection (Wyatt et al., J. Virol. 72:1725-1730 (1998)).

The development of small-molecule inhibitors directed against specific viral targets has become a focus of anti-HCV research. The determination of crystal structures for NS3 protease, e.g., Kim et al., Cell 87:343-355 (1996) and Love et al., Cell 87:331-342 (1996), and NS3 RNA helicases, e.g., Kim et al., Structure 6:89-100 (1998), has provided important structural insights for rational design of specific inhibitors.

Despite advances in understanding the genomic organization of the virus and the functions of viral proteins, fundamental aspects of HCV replication and pathogenesis remain unknown. A major challenge in gaining experimental access to HCV replication is the lack of an efficient cell culture system that allows production of infectious virus particles. Although infection of primary cell cultures and certain human cell lines has been reported, the amounts of virus produced in those systems and the levels of HCV replication have been too low to permit detailed analyses.

The construction of selectable subgenomic HCV RNAs that replicate with minimal efficiency in the human hepatoma cell line Huh-7 has been reported. Lohman et al. reported the construction of a replicon (I₃₇₇/NS3-3′) derived from a cloned full-length HCV consensus genome (genotype 1b) by deleting the C-p7 or C-NS2 region of the protein-coding region. Lohman et al., Science 285: 110-113 (1999). The replicon contained the following elements: (i) the HCV 5′ NTR fused to 12 amino acids of the capsid encoding region; (ii) the neomycin phosphtransferace gene (NPTII); (iii) the IRES from encephalomyocarditis virus (EMCV), inserted downstream of the NPTII gene and which directs translation of HCV proteins NS2 or NS3 to NS5B; and (iv) the 3′ NTR. After transfection of Huh-7 cells, only those cells supporting HCV RNA replication amplified the NPTII protein and developed resistance against the drug G418. While the cell lines derived from such G418 resistant colonies contained substantial levels of replicon RNAs and viral proteins, only 1 in 10⁶ transfected Huh-7 cells supported HCV replication.

Similar selectable HCV replicons were constructed based on an HCV-H genotype 1a infectious clone (Blight et al., Science 290:1972-74 (2000)). The HCV-H derived replicons were unable to establish efficient HCV replication, suggesting that the earlier-constructed replicons of Lohmann (1999), supra, were dependent on the particular genotype 1 b consensus cDNA clone used in those experiments. Blight et al. (2000), supra, reproduced the construction of the replicon made by Lohmann et al. (1999), supra, by carrying out a PCR-based gene assembly procedure and obtained G418-resistant Huh-7 cell colonies. Independent G418-resistant cell clones were sequenced to determine whether high-level HCV replication required adaptation of the replicon to the host cell. Multiple independent adaptive mutations that cluster in the HCV nonstructural protein NS5A were identified. The mutations conferred increased replicative ability in vitro, with transduction efficiency ranging from 0.2 to 10% of transfected cells as compared to earlier-constructed replicons in the art, e.g., the I₃₇₇/NS3-3′ replicon had a 0.0001% transduction efficiency.

Other reports, e.g., Krieger et al., J. Virol. 75, 4614-4624 (2001), have identified replicons with adaptive mutations. The replicon with best efficiency for G418 resistant colony formation was found to have several mutations in NS3, NS4B and NS5A. Out of these adaptive mutations, two mutations in NS3 (E1202G and T1280I) and one in NS5A (S2197P) were found to be sufficient to confer the higher replication (colony formation) phenotype.

The recent construction of selectable replicons and their transfection into mammalian cell lines has provided important insight into the replicon processes of HCV. Nonetheless, there is still a need for HCV reporter-selectable replicons that simplify the detection of HCV replication by monitoring reporter gene activity. An improved reporter-selectable HCV replicon would advance the ability to monitor HCV replication in these cell lines and would have a significant impact on accelerating HCV drug discovery research.

### SUMMARY OF THE INVENTION

The invention pertains to the construction of a reporter-selectable subgenomic HCV RNA (replicon) that replicates with high-levels of efficiency in the human hepatoma cell line Huh-7. In particular, a replicon has been constructed containing a humanized Renilla luciferase gene separated from an NPTII gene by a self-cleaving peptide of foot and mouth disease virus (FMDV) 2A proteinase. The replicon has two adaptive mutations in NS3 (E1202G and T1280I) and one in NS5A (S2197P). The Huh-7 cell line carrying the reporter-selectable replicon was found to have a highly efficient reporter gene signal (indicative of HCV RNA replication) over 20 passages. It was found to be very sensitive to known HCV inhibitors (e.g., interferon, IFN) with an EC₅₀, as determined by monitoring reporter gene signal, as compared to those obtained from other replicon cell lines.

In one general aspect, the invention is directed to Huh-7 cell line stably transformed with a nucleic acid molecule containing (i) an HCV 5′ NTR fused to the N-terminus of a capsid coding region; (ii) a humanized Renilla luciferase (hRLuc) nucleic acid molecule encoding a functional Renilla luciferase polypeptide, wherein said humanized Renilla luciferase nucleic acid molecule is fused at its N-terminus to said capsid and at its C-terminus to a foot and mouth disease virus (FMDV) 2A proteinase, which is fused to a neomycin phosphotransferase (NPTII) gene, wherein the hRLuc2A-NPTII fusion, upon expression from the HCV replicon, is cleaved at the 2A peptide to generate the hRLuc protein and the subsequent hRLuc signal and the NPTII protein that confers resistance to G418; (iii) an internal ribosome entry site (IRES) from encephalomyocarditis virus (EMCV), inserted downstream of the NPTII gene, which directs translation of HCV proteins NS3 to NS5B; and (iv) an HCV NS3-5B polyprotein coding region containing adaptive mutations. The adaptive mutations in the HCV NS3-5B polyprotein-coding region are selected from the group containing E1202G, T1280I and S2197P.

The invention is also directed to a cell line as described above, wherein the nucleic acid molecule is a self-replicating RNA molecule of BB7M4hRLuc, described in SEQ ID NO. 1.

The invention is further directed to a HCV double-stranded cDNA, which can be transcribed in vitro to produce replicating HCV RNA transcripts, wherein the cDNA contains a nucleic acid molecule containing (i) an HCV 5′ NTR fused to the N-terminus of a capsid coding region; (ii) a humanized Renilla luciferase (hRLuc) nucleic acid molecule encoding a functional Renilla luciferase polypeptide, wherein said humanized Renilla luciferase nucleic acid molecule is fused at its N-terminus to said capsid and at its C-terminus to a foot and mouth disease virus (FMDV) 2A proteinase, which is fused to a neomycin phosphotransferase (NPTII) gene, wherein the hRLuc2A-NPTII fusion, upon expression from the HCV replicon, is cleaved at the 2A peptide to generate the hRLuc protein and the subsequent hRLuc signal and the NPTII protein that confers resistance to G418; (iii) an internal ribosome entry site (IRES) from encephalomyocarditis virus (EMCV), inserted downstream of the NPTII gene, which directs translation of HCV proteins NS3 to NS5B; and (iv) an HCV NS3-5B polyprotein coding region containing adaptive mutations. The adaptive mutations in the HCV NS3-5B polyprotein-coding region are selected from the group containing E1202G, T1280I and S2197P.

The invention further provides an HCV double-stranded cDNA as described above, wherein the hRLuc2A-NPTII fusion upon expression is cleaved at the 2A peptide to generate the hRLuc protein and the subsequent hRLuc signal and the NPTII protein that confers resistance to G418. The cDNA described above may be present in a cell, and preferably in a prokaryotic cell.

The invention also provides methods of generating an Huh-7 cell line stably replicating an HCV hRLuc-selectable subgenomic replicon RNA, employing the steps of: (a) constructing a nucleic acid molecule comprising RNA sequences encoding the HCV replicon, wherein the nucleic acid molecule contains (i) an HCV 5′ NTR fused to the N-terminus of a capsid coding region; (ii) a humanized Renilla luciferase (hRLuc) nucleic acid molecule encoding a functional Renilla luciferase polypeptide, wherein said humanized Renilla luciferase nucleic acid molecule is fused at its N-terminus to said capsid and at its C-terminus to a foot and mouth disease virus (FMDV) 2A proteinase, which is fused to a neomycin phosphotransferase (NPTII) gene, wherein the hRLuc2A-NPTII fusion, upon expression from the HCV replicon, is cleaved at the 2A peptide to generate the hRLuc protein and the subsequent hRLuc signal and the NPTII protein that confers resistance to G418; (iii) an internal ribosome entry site (IRES) from encephalomyocarditis virus (EMCV), inserted downstream of the NPTII gene, which directs translation of HCV proteins NS3 to NS5B; and (iv) an HCV NS3-5B polyprotein coding region containing adaptive mutations; and (b) stably transforming Huh-7 cells with the nucleic acid molecule from step (a). The adaptive mutations in the HCV NS3-5B polyprotein-coding region are selected from the group containing E1202G, T1208I and S2197P.

The invention further provides an assay using a mammalian cell line derived by transfection of a reporter-selectable HCV replicon according to claim 10, wherein the assay is used to determine the specific antiviral activity of inhibitors in standard dose responses by using reporter gene activity as an endpoint. The cell line for the assay may be the Huh-7 cell line described herein, and the reporter gene activity may be from hRLuc, as described herein.

The invention is further directed to host cells containing the entire or part of the nucleic acid molecule of the invention. Examples of suitable host cells are Huh-7 cells, HeLa cells, VERO cells, CHO cells, COS cells, BHK cells, HEPG2 cells, 3T3 cells, or HEK293 cells.

The nucleic acid molecules and cell lines of the invention may also be used in a kit.

Other features and advantages of the invention will be apparent from the description that follows, which illustrates the invention and its preferred embodiments.

### BRIEF DESCRIPTION OF THE DRAWINGS

FIG. 1 depicts a schematic of the HCV genome and the HCV hRLuc-selectable replicon construct (BB7M4hRLuc). The 5′ and 3′ nontranslated regions (NTRs) flank the open reading frame with the structural proteins located in the NH₂-terminal portion of the polyprotein. The remainder encodes the nonstructural proteins (NS2 to NS5B). The reporter-selectable replicon, designated BB7-M4-hRLuc, has the 5′ NTR fused to a small portion of the core coding region, the humanized Renilla luciferase gene (hRLuc), a self-cleaving peptide of foot and mouth disease virus (FMDV) 2A proteinase, the NPTII gene, and an EMCV IRES (designated “EI”), followed by the NS3 to NS5B HCV coding region and the 3′ NTR region.

FIG. 2 depicts a flow chart of the process for constructing the HCV hRLuc-selectable replicon (BB7M4hRLuc). FIG. 2A: Mutations I2204S, E1202G, T1208I and S2197P were introduced into the BB7 plasmid by PCR based mutagenesis (nucleic acid changes, shown). The SspBI-XhoI restriction endonuclease fragment from the mutated BB7 plasmid was substituted with that of the original, unmutated BB7 plasmid, resulting in BB7-M4. FIG. 2B: Two polymerase chain reactions (PCR) were used to generate the fusion between the hRLuc gene and the FMDV 2A self-cleaving peptide. The final PCR product had an AscI site at the 5′ end and FMDV 2A fused to the hRLuc at the 3′ end, followed by the AscI site. This PCR product was cloned into an AscI site of a plasmid that contained an XbaI-PmeI fragment of BB7 (pcDNA3.1-HCVIRESNeo). This resulted in a fusion of the 36 nucleotides of the HCV core protein with the ORF of hRLuc that was fused to the NPTII coding region via the FMDV 2A peptide (in pcDNA3.1-HCVIRES-hRLuc2A Neo). FIG. 2C: The pcDNA3.1-HCVIRES-hRLuc2A Neo construct and BB7-M4 were digested with AgeI and PmeI. The small AgeI-PmeI fragment from pcDNA3.1-HCVIRES-hRLuc2A Neo construct was ligated with the large AgeI-PmeI fragment of BB7M4, resulting in the construction of BB7M4hRLuc.

FIG. 3 depicts the nucleotide sequence of BB7M4hRLuc (SEQ ID NO: 1).

FIG. 4 depicts data obtained to validate Huh-7 cell line BB7M4hRLuc#10.

FIG. 5 depicts a comparison of actual luciferase activity in terms of relative light units (RLU), which is indicative of HCV RNA replication and a signal to noise ratio of Huh-7 cell line BB7M4hRLuc#10 with that of the other available reporter-replicon line.

FIG. 6 depicts the nucleotide sequence of BB7 (SEQ ID NO: 2).

### DETAILED DESCRIPTION OF THE INVENTION AND PREFERRED EMBODIMENTS

The invention pertains to the construction of a selectable subgenomic HCV replicon RNA that contains a reporter gene and is capable of a high level of replication in the human hepatoma cell line Huh-7, as displayed by a large signal to noise ratio as compared to available reporter-selectable replicons. In particular, a replicon has been constructed that contains a humanized Renilla luciferase gene separated from a NPTII gene by a self-cleaving peptide of foot and mouth disease virus 2A proteinase. The Huh-7 cell line carrying the reporter-selectable replicon was found to have a stable reporter gene signal over 20 passages and sensitivity to known HCV inhibitors with inhibition values (EC₅₀) comparable to those obtained from other replicon cell lines.

As used herein, the terms “comprising” and “including” are used in an open, non-limiting sense.

In accordance with the present invention there may be employed conventional molecular biology, microbiology, and recombinant DNA techniques within the skill of the art. Such techniques are explained fully in the literature. See, e.g., Maniatis et al., “Molecular Cloning: A Laboratory Manual,” (1989); Ausubel, Ed., “Current Protocols in Molecular Biology,” Volumes I-III (1994); Celis, Ed., “Cell Biology: A Laboratory Handbook,” Volumes I-III (1994); Coligan, Ed., “Current Protocols in Immunology,” Volumes I-III (1994); Gait, Ed., “Oligonucleotide Synthesis” (1984); Hames et al., Eds., “Nucleic Acid Hybridization” (1985); Hames et al., “Transcription and Translation” (1984); Freshney, Ed., “Animal Cell Culture” (1986); IRL Press, “Immobilized Cells and Enzymes” (1986); and Perbal, “A Practical Guide To Molecular Cloning” (1984).

Therefore, if appearing herein, the following terms shall have the definitions set out below.

“Polynucleotide” or “nucleic acid molecule” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

In addition, the term “DNA molecule” refers only to the primary and secondary structure of the molecule, and does not limit it to any particular tertiary forms. Thus, the term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “RNA molecule” refers to the polymeric form of ribonucleotides in its either single-stranded form or a double-stranded helix form. In discussing the structure of particular RNA molecules, sequence may be described herein according to the normal convention of giving the sequence in the 5′ to 3′ direction.

Amino acid residues described herein are preferred to be in the “L” isomeric form. However, residues in the “D” isomeric form can be substituted for any L-amino acid residue, as long as the desired functional property is retained by the polypeptide.

The term NH₂ refers to the free amino group present at the amino terminus of a polypeptide, while COOH refers to the free carboxy group present at the carboxy terminus of a polypeptide. Standard polypeptide nomenclature and abbreviations for amino acid residues are used herein.

Amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues.

A “replicon” is any genetic element (e.g., plasmid, chromosome, viral RNA) that functions as an autonomous unit of DNA or RNA replication in vivo. That is, it is capable of replication under its own control. Bradenbeck et al., Semin. Virol. 3:297-310 (1992).

A “vector” is a circular DNA, such as a plasmid, phage or cosmid, to which another DNA segment may be attached so as to bring about the replication, expression or integration of the attached segment.

A variety of expression vectors can be used to express a nucleic acid molecule. Such vectors include chromosomal, episomal, and virus-derived vectors, e.g., vectors derived from bacterial plasmids, from bacteriophage, from yeast episomes, from yeast chromosomal elements, including yeast artificial chromosomes, from viruses such as baculoviruses, papovaviruses such as SV40, vaccinia viruses, adenoviruses, poxviruses, pseudorabies viruses, herpes viruses, and retroviruses. Vectors may also be derived from combinations of these sources, such as those derived from plasmid and bacteriophage genetic elements, e.g., cosmids and phagemids. Appropriate cloning and expression vectors for prokaryotic and eukaryotic hosts are described in Sambrook et al., supra.

A vector containing the appropriate nucleic acid molecule can be introduced into an appropriate host cell for propagation or expression using known techniques. Host cells can include bacterial cells including, but not limited to, E. coli, Streptomyces, and Salmonella typhimurium, eukaryotic cells including, but not limited to, yeast, insect cells, such as Drosophila, animal cells, such as Huh-7, HeLa, COS, HEK 293, MT-2T, CEM-SS, and CHO cells, and plant cells.

Vectors generally include selectable markers that enable the selection of a subpopulation of cells that contain the recombinant vector constructs. The marker can be contained in the same vector that contains the nucleic acid molecules described herein or may be on a separate vector. Markers include tetracycline- or ampicillin-resistance genes for prokaryotic host cells and dihydrofolate reductase or neomycin resistance for eukaryotic host cells. However, any marker that provides selection for a phenotypic trait will be effective.

A “coding sequence” or “open reading frame” is a nucleotide sequence that is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA or RNA sequences.

Transcriptional control sequences are DNA regulatory sequences, such as promoters, enhancers, polyadenylation signals, terminators, and the like, that provide for the expression of a coding sequence in a host cell by synthesis of messenger RNA (mRNA) from the DNA template.

A “promoter sequence” is a DNA regulatory region capable of binding RNA polymerase in a cell and initiating transcription of a coding sequence. For purposes of defining the present invention, a promoter sequence is bound at its 3′ terminus by the transcription initiation site and extends upstream (5′ direction) to include the minimum number of bases or elements necessary to initiate transcription at levels detectable above background. Within the promoter sequence will be found a transcription initiation site, conveniently defined by mapping with nuclease S1, as well as protein binding domains (consensus sequences) responsible for the binding of RNA polymerase. Prokaryotic promoters contain −10 and −35 consensus sequences. A promoter can also be used to refer to RNA sequences or structures in RNA virus replication.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. RNA sequences can also serve as expression control sequences by virtue of their ability to modulate translation, RNA stability, and replication (for RNA viruses).

A “signal sequence” can be included before the coding sequence. This sequence encodes a signal peptide, N-terminal to the polypeptide, which communicates to the host cell to direct the polypeptide to the cell surface or secrete the polypeptide into the media. The signal peptide is clipped off by the host cell before the protein leaves the cell. Signal sequences can be found associated within a variety of proteins native to eukaryotes.

The term “oligonucleotide” is defined as a molecule comprised of two or more deoxyribonucleotides, preferably more than three. Its exact size will depend upon many factors, which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced. That is, inducement in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. The primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby carry out the synthesis of the extended product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA or RNA when such DNA or RNA has been introduced inside the cell. The transforming DNA or RNA may or may not be integrated (covalently linked) into chromosomal DNA making up the genome of the cell. For example, in prokaryotes, yeast, and mammalian cells, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. In the case of an RNA replicon that transforms a mammalian cell as described in the present invention, the RNA molecule, e.g., an HCV RNA molecule, has the ability to replicate semi-autonomously. Huh-7 cells carrying the HCV replicons get selected in the presence of G418 since HCV RNA replication results in resistance to G418 by production of the neomycin phosphotransferase protein. This results in clones of Huh-7 cells resistant to G418, which are capable of forming cell lines.

A “clone” is a population of cells derived from a single cell or common ancestor by mitosis.

The term “recombinant host cell” refers to a cell that has been altered to contain a new combination of genes or nucleic acid molecules. The recombinant host cells were prepared by introducing the vector constructs described herein into the cells by techniques readily available in the art. These include calcium phosphate transfection, DEAE-dextran-mediated transfection, cationic lipid-mediated transfection, electroporation, transduction, infection, lipofection, and other techniques, such as those found in Sambrook et al., supra.

Host cells can contain more than one vector. Thus, different nucleotide sequences can be introduced on different vectors to the same cell. Similarly, the nucleic acid molecules can be introduced either alone or with other nucleic acid molecules that are not related to the nucleic acid molecules, such as those providing trans-acting factors for expression vectors. When more than one vector is introduced into a cell, the vectors can be introduced independently, co-introduced or segments of each vector can be combined into one vector. The invention also relates to recombinant host cells containing the vectors described herein.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

A “cell line” is a clone of a primary cell that is capable of stable growth in vitro for many generations. This definition can be applied to RNA molecules, which can be used to transform or “transfect” cells. For some RNA viruses, such methods can be used to produce cell lines which transiently or continuously support virus replication and, in some cases, which produce infectious viral particles.

Two DNA or RNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al, supra.

A “heterologous” region of a DNA or RNA construct is an identifiable segment of DNA or RNA molecule within a larger nucleic acid that is not found in association with the larger molecule in nature. For instance, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG or AUG) in front of the DNA sequence to be expressed and maintaining the correct reading frame to permit expression of the DNA sequence under the control of the expression control sequence and production of the desired product encoded by the DNA sequence. If a gene to be inserted into a recombinant DNA molecule does not contain an appropriate start signal, such a start signal can be inserted in front of the gene.

The term “standard hybridization conditions” in general refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of standard hybridization conditions is whether the two sequences hybridizing are RNA-RNA, DNA-DNA or RNA-DNA. Such standard hybridization conditions are easily determined by one skilled in the art according to well-known formulae, wherein hybridization is typically 10-20° C. below the predicted or determined Tₘ.

As used herein, “pg” means picogram, “ng” means nanogram, “ug” or “μg” mean microgram, “mg” means milligram, “ul” or “μl” mean microliter, “ml” means milliliter, “l” means liter, “min.” means minutes and “sec.” means seconds.

Hepatitis C virus or HCV refers to a diverse group of related viruses classified as a separate genus in the Flaviviridae family. The characteristics of this genus are described in the Background of the Invention above, and include such members as HCV-1, HC-J1, HCV-J, HCV-BK, HCV-H, HC-J6, HC-J8, HC-J4/83, HC-J4/91, HC-C2, HCV-JK1, HCV-T, HCV-JT, HC-G9, and the like.

HCV analogs may be prepared from nucleotide sequences derived within the scope of the present invention. Analogs, such as fragments or mutants can be produced by standard cleavage by restriction enzymes, or site-directed mutagenesis of the HCV coding and non-coding (5′ and 3′ terminal) sequences. Molecules exhibiting “HCV inhibiting activity” such as small molecules or antisense molecules may be identified by assays, e.g., using interferon.

Replication of HCV in cells can be ascertained by branched TaqMan quantitative RT/PCR and immunological procedures. The procedures and their application are well known in the art and accordingly may be utilized within the scope of the present invention. A “competitive” antibody binding procedure is described in U.S. Pat. Nos. 3,654,090 and 3,850,752. A “sandwich” procedure is described in U.S. Pat. Nos. RE 31,006 and 4,016,043. Still other procedures are known such as the “double antibody”, or “DASP” procedure.

In each instance, HCV proteins form complexes with one or more antibodies or binding partners and one member of the complex is labeled with a detectable label. The fact that a complex has formed and, if desired, the amount thereof, can be determined by known methods applicable to the detection of labels.

Alternatively, the presence of HCV RNA can be determined by Northern analysis, primer extension, and the like. The labels most commonly employed for these studies are radioactive elements, enzymes that fluoresce when exposed to substrate and others. A number of fluorescent materials are known and can be utilized as labels. These include, for example, fluorescein, rhodamine, auramine, Texas Red, AMCA blue and Lucifer Yellow.

An antibody to HCV proteins or a probe for HCV RNA can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

Enzyme labels are likewise useful, and can be detected by any of the presently utilized colorimetric, spectrophotometric, fluorospectrophotometric, techniques. The enzyme is conjugated to the selected probe by reaction with bridging molecules such as carbodiimides, diisocyanates, glutaraldehyde and the like. Many enzymes that can be used in these procedures are known and can be utilized. Those preferred are peroxidase, beta-glucuronidase, beta-D-glucosidase, beta-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods. In addition, a probe may be biotin-labeled, and thereafter be detected with labeled avidin, or a combination of avidin and a labeled anti-avidin antibody. Probes may also have digoxygenin incorporated therein and be then detected with a labeled anti-digoxygenin antibody.

An EC₅₀ value is the concentration of the inhibitor at which 50% inhibition of viral replication is achieved. An HCV replicon reporter assay system can be developed to determine the specific antiviral activity of inhibitors in standard dose response assays. In such assays, the reporter-selectable containing Huh-7 cells are incubated in 96 wells containing serial dilutions of test inhibitors or no inhibitor. At a specified time after incubation, the activities of the viral-encoded reporter genes are measured in the cell lines using the appropriate reporter assay methodologies. Data from the reporter gene measurements can be expressed as the percent of reporter gene activity in inhibitor-treated cells relative to that of inhibitor-free cells. An analysis of the antiviral component of such data allows for the calculation of the fifty-percent effective concentration (EC₅₀).

An internal ribosomal entry site (IRES) recruits ribosomes in a cap-independent manner to carry out translation. The HCV RNA genome contains an internal ribosome entry site.

A “BB7” construct (obtained from Apath, L.L.C., St. Louis, Mo.) is a subgenomic HCV RNA (replicon) having one adaptive mutation (S2204I) in the NS5A domain.

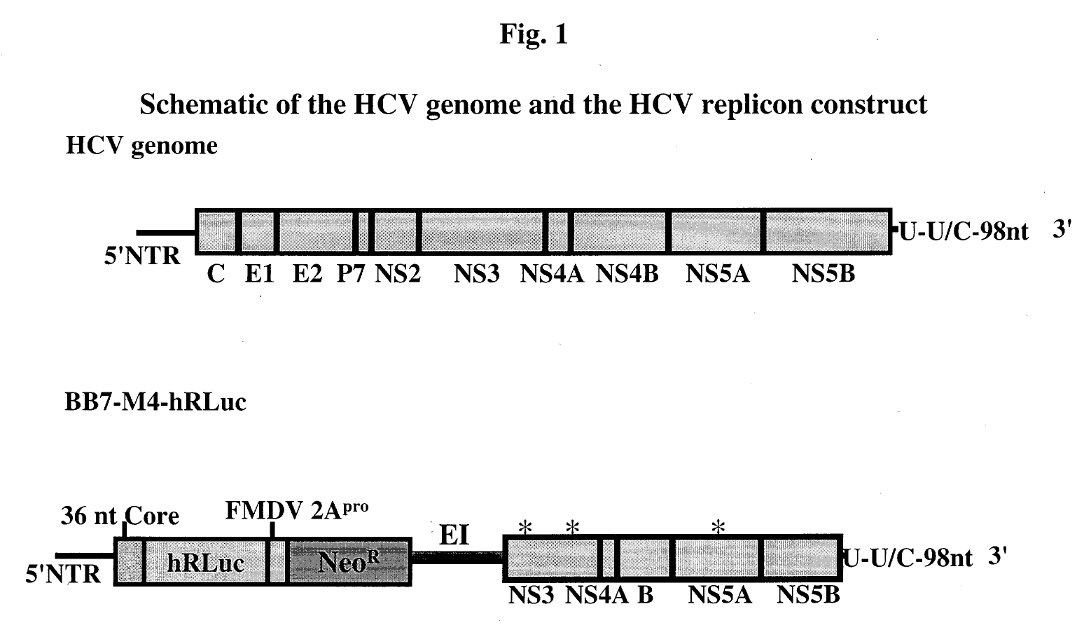

The reporter-selectable HCV replicon, which may be designated herein BB7M4hRLuc, has a reporter gene to monitor HCV replication and three adaptive mutations, two in the NS3 domain and one in the NS5A domain (FIG. 1). The reporter gene (hRLuc) is fused to the NPT II gene via a self-cleaving peptide encoding the 2A proteinase of FMDV. This fusion protein is under the translational control of the HCV IRES residing in the 5′ nontranslated region (NTR) of HCV RNA. The second cistron of the replicon that comprises the HCV nonstructural protein region from NS3-NS5B is under the translational control of EMCV IRES.

In cell lines containing the BB7M4hRLuc2A replicon, an increase in replicon RNA by HCV replication results in an increase in hRLuc and NPTII protein production. The high activity of the former can be detected in the replicon cell line by adding a substrate, and the NPTII activity results in stable colony and cell line formation.

The present invention is an improvement over other HCV-based reporter-selectable replicons in the art in that the signal to noise ratio of the current invention is seventy-fold higher compared to what has been disclosed in the art.

### Exemplary Methods and Materials

HCV Replicon Construction

A. Humanized Renilla Luciferase

The reporter-selectable replicon of the present invention contains a humanized Renilla luciferase gene (hRLuc) that serves as a reporter gene.

Reporter genes are used throughout the biological sciences as a means to identify and analyze regulatory elements of genes. Using recombinant DNA techniques, reporter genes can be fused to a regulatory sequence of interest. The resulting recombinant gene is then introduced into cells where the expression of the reporter can be detected using various methods, including measurement of the reporter mRNA, measurement of the reporter protein, or measurement of the reporter enzymatic activity. Commonly used reporter genes include beta-galactosidase, firefly luciferase, bacterial luciferase, Renilla luciferase, alkaline phosphatase, chloramphenicol acetyltransferase (CAT), green fluorescent protein (GFP) and beta-glucuronidase (GUS).

Many reporter systems utilize luciferase genes. Luciferase refers to a group of enzymes that catalyze the oxidation of various substrates to produce a light emission. Generally, luciferase activity is not found in eukaryotic cells. The different luciferases have different specific requirements and may be used to detect and quantify a variety of substances.

The wild-type luciferase enzyme of the sea pansy Renilla reniformis is a monomeric protein with a molecular weight of 36 kDa. This enzyme catalyzes the emission of visible light in the presence of oxygen and the luciferin coelenterazine to produce blue light. The luciferase gene from Renilla has been used to assay gene expression in bacterial (Jubin et al., Biotechniques 24:185-188 (1998)), yeast (Srikantha et al., J. Bacteriol. 178:121-129 (1996)), plant (Mayerhofer et al., Plant J. 7:1031-1038 (1995)), and mammalian cells (Lorenz et al., J. Biolumin. Chemilumin. 11:31-37 (1996)).

The cloning, expression and use of wild-type Renilla luciferase are reported in U.S. Pat. Nos. 5,292,658 and 5,418,155.

Renilla luciferase is available commercially (Boehringer Mannheim, Sigma, and Promega). Promega (Madison, Wis.) has developed a synthetic Renilla luciferase gene that contains codons optimized for efficient expression in mammalian cells. Literature from Promega indicates that additional features of this modified gene include removal of potentially interfering restriction sites and genetic regulatory sites from the gene (Promega Technical Manual No. 055, revised Jun. 1, 2001). Sequence information related to various plasmids containing the Promega humanized Renilla luciferase gene is deposited with GenBank under accession numbers AF362545-AF362551.

The term “functional derivative” with respect to a polypeptide is a polypeptide that possesses a biological activity (either functional or structural) or an immunological characteristic that is substantially similar to a biological activity or an immunological characteristic of the humanized Renilla luciferase described herein.

B. Adaptive Mutations

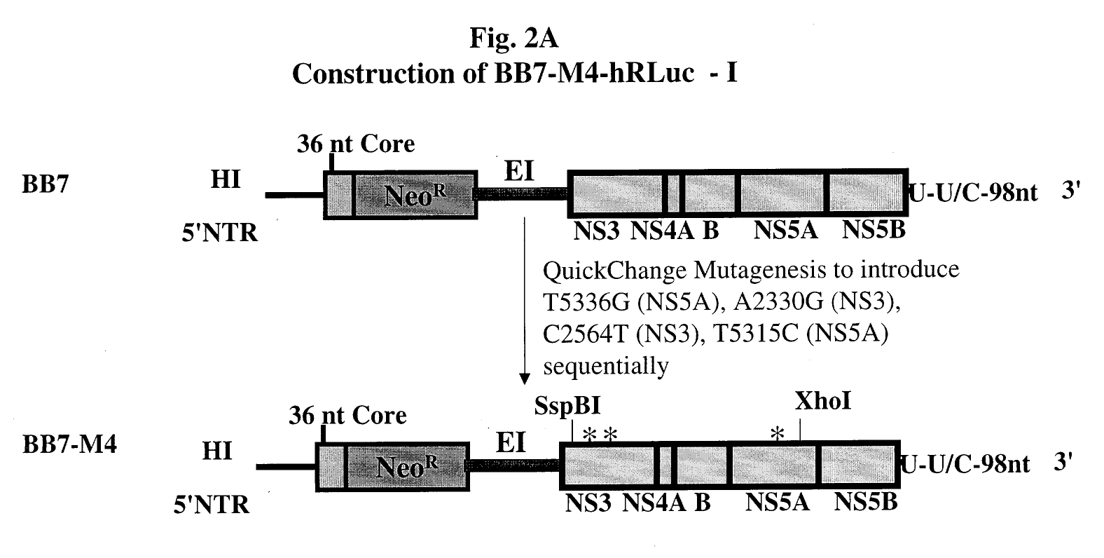

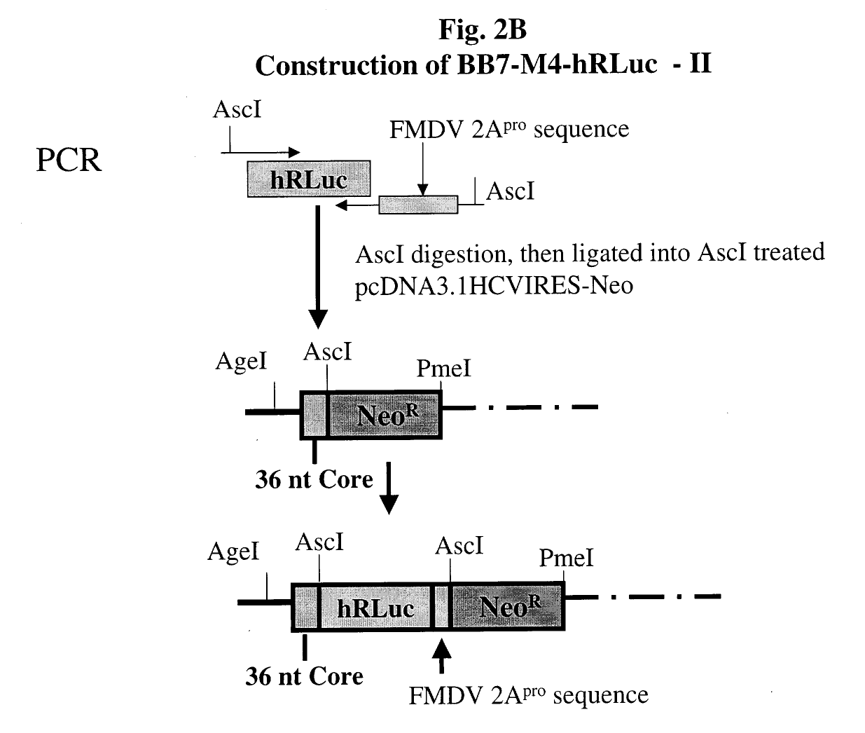

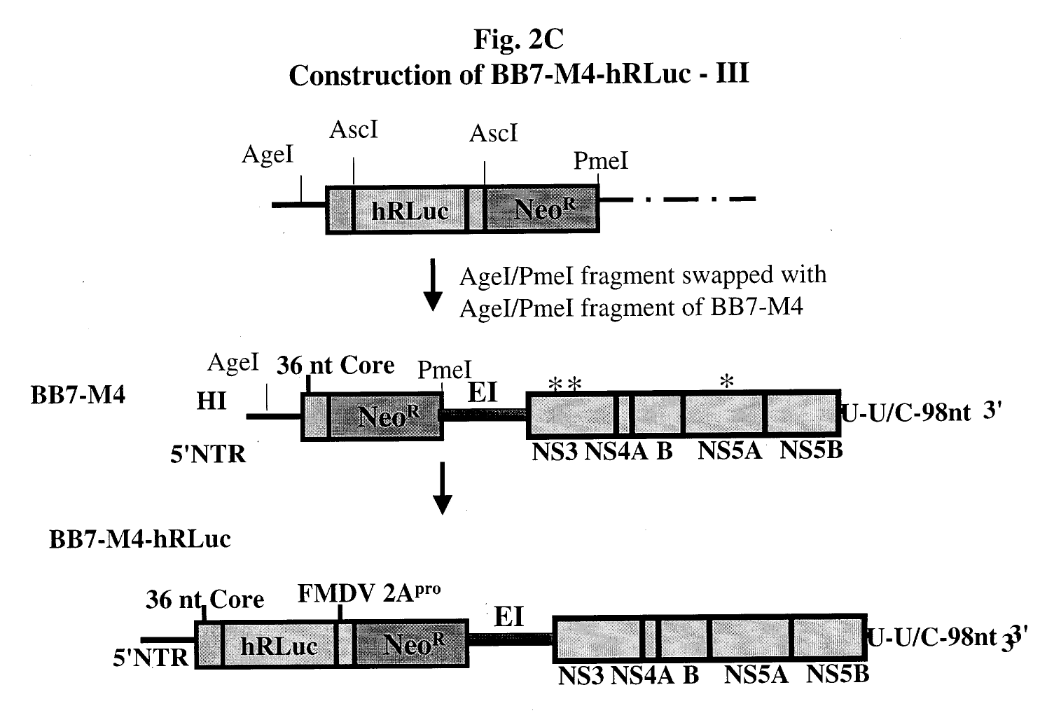

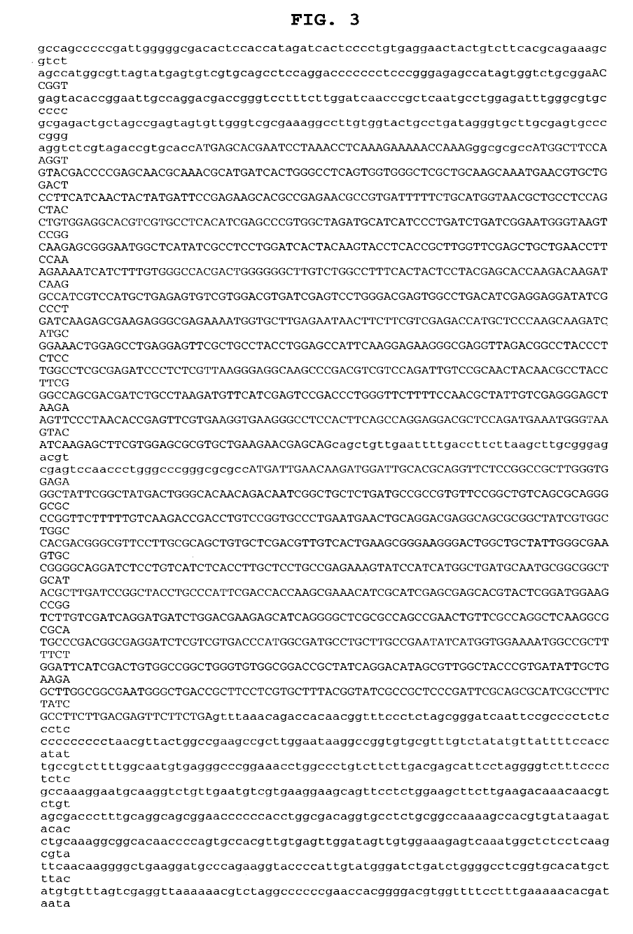

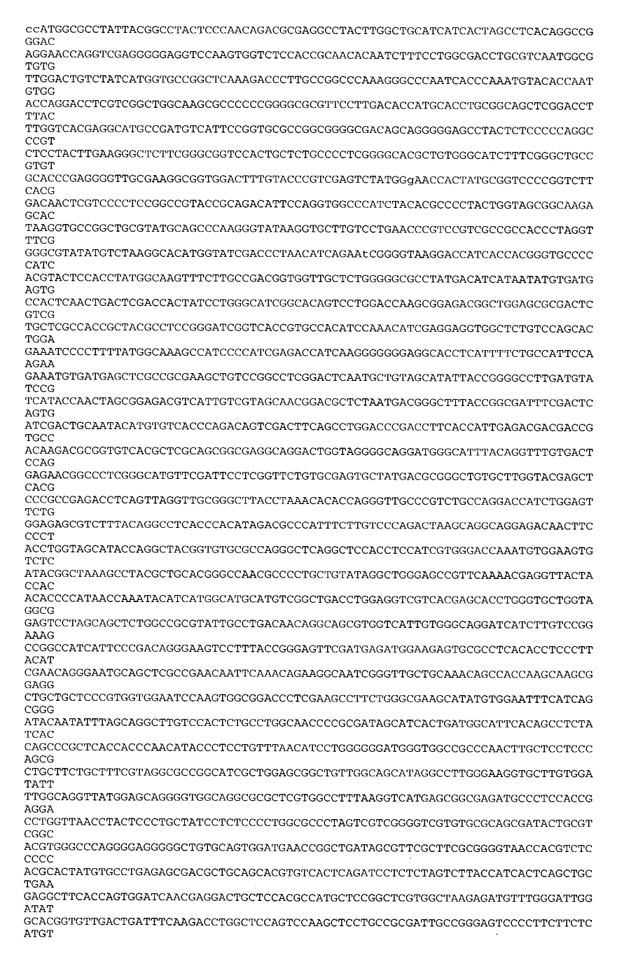

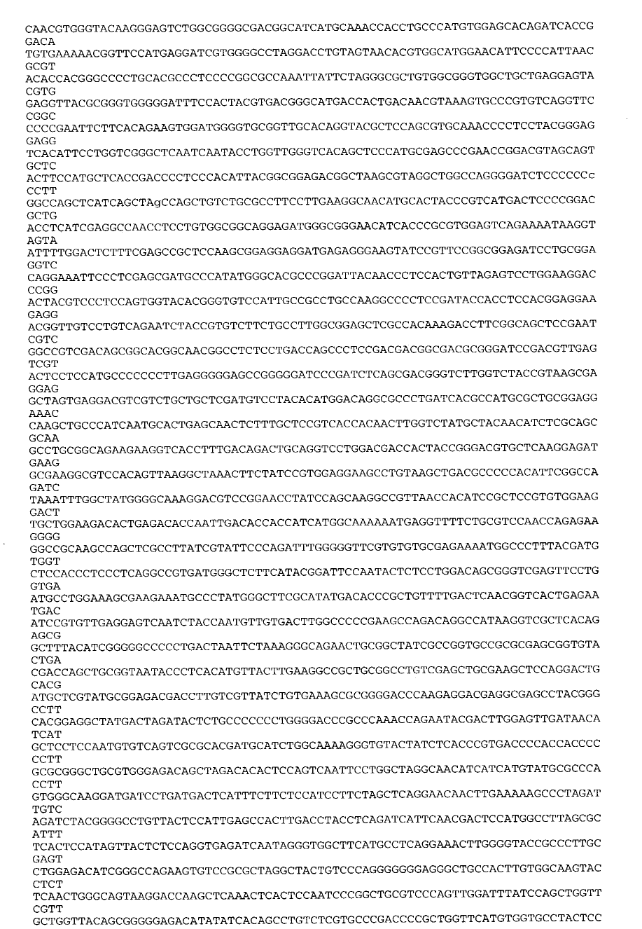

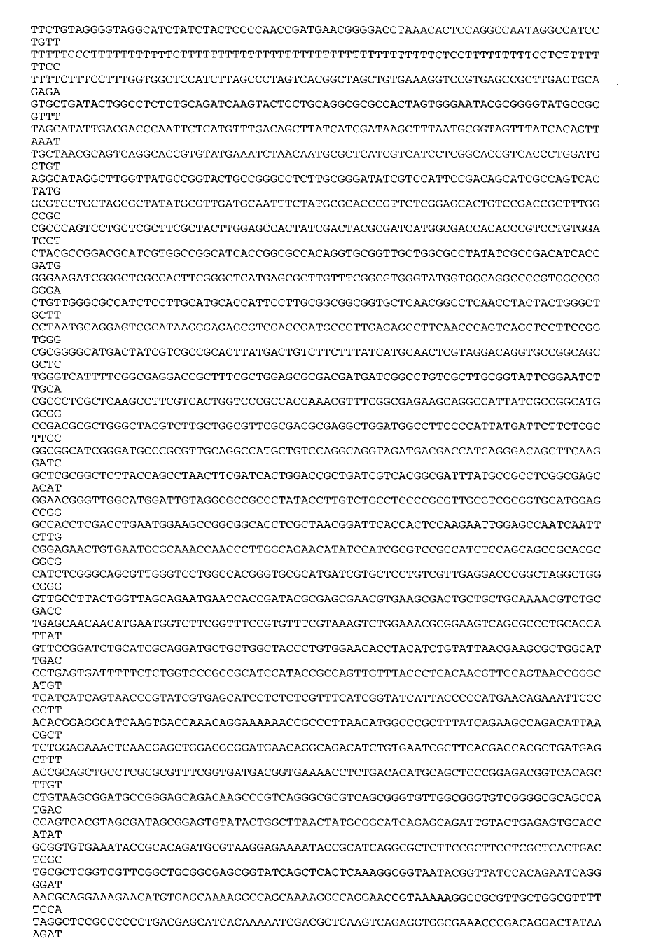

Mutations I2204S, E1202G, T1208I and S2197P were introduced into BB7 by quick-change site-directed PCR based mutagenesis. (FIG. 2A, Stratagene, La Jolla; Wang et al., “BioTechniques,” Vol. 26, No. 4 (1999); Kunkel, T. A., Proc. Natl. Acad. Sci, USA 82:488 (1985); Vandeyar et al., Gene 65:129-133 (1988); Sugimoto et al., Anal. Biochem. 179:309-311 (1989); Taylor et al., Nucleic Acids Res. 13:8764 (1985); Papworth et al., Structure 9:3-4 (1996); Bergseid, et al., Structure 4:34-35 (1991); Nelson et al., Methods Enzymol. 216:279-303 (1992); and Burke et al., Oncogene 16:1031-1040 (1998).

The BB7 NS5A mutation S2204I was changed back to the wild type sequence (I2204S). The primers used for making the four amino acid changes are described below in Table 1.

TABLE 1

| Name of | |
|---|---|
| the Primers | Sequences of Primers |
| BB7-I2204S(+) | 5′-GCC AGC TCA TCA GCT AGC CAG CTG |
| (SEQ ID NO. 3) | TCT GCG CC-3′ |
| BB7-I2204S(−) | 5′-GCG CAG ACA GCT GGC TAG CTG ATG |
| (SEQ ID NO. 4) | AGC TGG C-3′ |
| BB7-E1202G(+) | 5′-GGA CTT TGT ACC CGT CGA GTC TAT |
| (SEQ ID NO. 5) | GGG AAC CAC TAT GCG GTC CCC GGT CTT |
| | CAC G-3′ |
| BB7-E1202G(−) | 5′-CGT GAA GAC CGG GGA CCG CAT AGT |
| (SEQ ID NO. 6) | GGT TCC CAT AGA CTC GAC GGG TAC AAA |
| | GTC C-3′ |
| BB7-T1280I(+) | 5′-GGC ACA TGG TAT CGA CCC TAA CAT |
| (SEQ ID. NO.7) | CAG AAT CGG GGT AAG GAC CAT CAC CAC |
| | GGG TGC-3′ |
| BB7-T1280I(−) | 5′-GCA CCC GTG GTG ATG GTC CTT ACC |
| (SEQ ID NO. 8) | CCG ATT CTG ATG TTA GGG TCG ATA CCA |
| | TGT GCC-3′ |
| BB7-S2197P(+) | 5′-GCG TAG GCT GGC CAG GGG ATC TCC |
| (SEQ ID NO. 9) | CCC CCC CTT GGC CAG CTC ATC AGC TAG |
| | CCA GC-3′ |
| BB7-S2197P(−) | 5′-GCT GGC TAG CTG ATG AGC TGG CCA |
| (SEQ ID | AGG GGG GGG GAG ATC CCC TGG CCA GCC |
| NO. 10) | TAC GC-3′ |

To make the I2204S change in the BB7 construct, components were added into two thin-wall PCR tubes as listed below in Table 2.

TABLE 2

| Components | Volume (μl) |
|---|---|
| 10x Herculase Buffer | 5 |
| BB7-I1179S (5 ng/ul) | 40 |
| BB7 I2204S (+) or BB7 I2204S (−) primers (both 20 pm/ul) | 1 |
| 2 mM dNTPs (20 μM) | 1.25 |
| ddH₂O | 1.75 |
| Herculase | 1 |

Polymerase chain reactions were carried out for 3 cycles at 95° C. for 30 seconds, then 55° C. for 1 minute, and 68° C. for 26 minutes. Following the completion of the extension reactions, 25 μl from both reactions were mixed and 1 μl Herculase (or PFU) added before subjecting the reaction to 12 cycles at 95° C. for 30 seconds, then 55° C. for 1 minute and 68° C. for 26 minutes. Two microliters of the finished PCR reaction was used to transform competent E. coli cells (Invitrogen, Carlsbad, Calif.). Clones were selected on tetracycline-containing plates (10 μg/ml). Positive clones were identified by restriction digestion. Then the point mutation was confirmed by sequencing analysis. The new construct was named BB7-M2.

The same strategy was employed to introduce the E1202G mutation, except BB7-M1 was used as the PCR template and primers BB7-E1202G(+) and BB7-E1202G(−) were used. The new construct was named BB7-M2. To make the BB7-M3-template, BB7-M2 was used as the PCR template, with BB7-T1280I(+) and BB7-T1280I(−) as the primers in the PCR reaction. Primers BB7-S2197P(+) and BB7-S2197P(−) were used in conjunction with BB7-M3 as the template to generate BB7-M4.

All mutations were confirmed by sequencing. In order to make sure that secondary mutations were not introduced during the PCR reaction, the SspBI-XhoI restriction endonuclease fragment from the mutated BB7-M4 plasmid was substituted for that of the original, unmutated BB7 plasmid, resulting in the final BB7-M4 plasmid. In order to carry out the substitution, the plasmid that was subjected to mutagenesis and BB7 were cut with the restriction enzymes SspBI and XhoI. The small SspBI-XhoI fragment from the mutagenized plasmid was gel purified and ligated with the large SspBI-XhoI fragment (also gel purified) of BB7. The resulting construct had only a fragment of the DNA that underwent mutagenesis, therefore rendering the construct free of inadvertent mutations elsewhere in the plasmid DNA that might have risen during the mutagenesis process.

C. Construction of BB7M4hRLuc (via hRLuc2A and hRLuc2ANPTII Fusions)

The luciferase gene of the invention can be used to construct fusion proteins. The construction of fusion proteins is known in the art, e.g., Day et al., Biotechniques 25:848-50, 852-4, 856 (1998); Kobatake et al., Anal Biochem. 208:300-305 (1993)); and Wang et al., Mol. Gen. Genet. 264:578-87 (2001)).

In order to generate the fusion between the hRLuc gene and the self-cleaving peptide of foot and mouth disease virus (FMDV), two polymerase chain reactions were employed. PCR techniques are well known in the art. See, e.g., Innis et al., Eds., “PCR Applications: Protocols for Functional Genomics” (1999); Gelfand et al., Eds., “PCR Protocols: A Guide to Methods and Applications” (1990); Freshney, Ed., “Animal Cell Culture” (1986); and Perbal, “A Practical Guide to Molecular Cloning” (1984).

Briefly, 50 μl PCR mixtures were prepared containing 50 pmol for each of the appropriate oligonucleotide primers, 20 ng of template DNA, a final concentration of 200 μM for each dNTP (Roche, Indianapolis, Ind.), 5 units of Herculase enhanced DNA polymerase (Stratagene, San Diego, Calif.), and 5 μl of the 10× Herculase enhanced DNA polymerase reaction buffer provided by the manufacturer (Stratagene). PCR reactions were initiated by incubation at 95° C. for 2 minutes. PCR amplification was then carried out for 30 iterative cycles with each cycle consisting of the following steps: (1) 30 seconds at 95° C.; (2) 1 minute at 60° C.; and (3) 1 minute at 72° C.

In the first PCR reaction, oligonucleotides AscI-hRLuc(+) and FMDV2A-hRLuc(−) were used to amplify the hRLuc gene from phRL-CMV, purchased from Promega (Madison).

The sequence of the primers for the PCR reaction were:

AscI-hRLuc(+):

5′-CCA ggc gcg ccA TGG CTT CCA AGG TGT ACG ACC CCG AGC-3′

(SEQ ID NO: 11). The AscI site (ggcgcgcc) is followed by 28 nts from the 5′ end of open reading frame (ORF) of hRLuc.

FMDV2A-hRLuc(−):

5′-gac tcg acg tct ccc gca agc tta aga agg tca aaa ttc aac agc tgC TGC TCG TTC TTC AGC ACG CGC TCC ACG-3′

(SEQ ID NO: 12). The lower case letters denote partial FMDV2A sequences, upper case is hRLuc sequences, and the stop codon is deleted.

The resulting PCR product has an AscI site at the 5′ end, and a partial FMDV 2Apro fused to the hRLuc gene. In the second PCR, the PCR product from the first PCR was used as the template, oligonucleotides AscI-hRLuc(+) and AscI-G-FMDV2A(−) were used as primers.

The sequence for AscI-G-FMDV2A(−) is:

5′-cca GGC GCG CCc ggg ccc agg gtt gga ctc gac gtc tcc cgc aag ctt aag aag gtc aaa att c

(SEQ ID NO: 13). (The extra C was inserted before AscI site (upper case) to keep the ORF in frame with the ORF of NPT II).

The final PCR product has the AscI site at the 5′ end, FMDV 2A fused to the hRLuc at the 3′end, followed by the AscI site (See FIG. 2B).

The PCR product was then digested with the restriction endonuclease AscI, and introduced into pcDNA3.1-HCVIRES-Neo, which contains the HCV IRES with partial Core fused to the NPT II gene (See FIG. 2B). The orientation of the hRLuc gene in pcDNA3.1-HCVIRES-hRLuc2ANeo was verified by PCR using a pair of primers in which a positive-sense primer corresponded to nt 332-361 of the HCV genome and a negative-sense primer annealed to the hRLuc gene. PCR products were only obtained from those clones that contain the correct orientation of hRLuc gene. After verifying the correct orientation of hRLuc gene in pcDNA3.1-HCVIRES-hRLuc2A Neo, this construct and BB7-M4 were digested with AgeI and PmeI. The small AgeI-PmeI fragment from pcDNA3.1-HCVIRES-hRLuc2A Neo was gel purified and ligated with the large, gel-purified AgeI-PmeI fragment of BB7M4. This resulted in the construction of BB7M4hRLuc (See FIG. 2C).

D. Cell Line Generation

Huh-7 cells (obtained from Apath, L.L.C.) were propagated in Dulbecco's Modified Eagle Medium (DMEM; Invitrogen, Carlsbad, Calif. (formerly Life technologies)) containing 10% fetal bovine serum (FBS, HyClone, Logan, Utah), 100 IU/ml of penicillin and 100 mg/ml of streptomycin sulfate (Invitrogen, Carlsbad, Calif.).

The BB7M4hRLuc cDNA was linearized by digestion with restriction nuclease ScaI. The DNA was purified by extracting with phenol-chloroform and precipitated with 100% ethanol. The linearized cDNA was used for carrying out in vitro transcription using the Megascript kit (Ambion, Austin, Tex.). Briefly, 1 μg of linearized cDNA was incubated with 2 μl each of the provided ATP, CTP, GTP and UTP solutions, 2 μl of 10× reaction buffer and 2 μl of the T7 bacteriophage RNA polymerase in a final volume of 20 μl, made up by nuclease free water. The reaction was incubated for an hour at 37° C. and treated with DNAse I based on the manufacturer's recommendations. In vitro synthesized RNA transcripts were purified using an RNeasy™ mini kit (Qiagen, Palo Alto, Calif.).

In one embodiment, replicon BB7M4hRLuc-in vitro-transcribed RNA was introduced into Huh-7 cells by electroporation to generate three cell lines.

Huh-7 cells were seeded at 4.1×10⁶ in separate T225 tissue culture flasks. The cells were incubated at 37° C., 5% CO₂, for approximately 24 hrs. Approximately two flasks were used for each electroporation.

The cells were collected by first removing the media from each flask and washing the cells once with phosphate-buffered saline (PBS). The PBS was then removed by aspiration. Three milliliters of Trypsin-EDTA were added to each flask, ensuring that all cells were covered by Trypsin-EDTA and then removed by aspiration. The cells were then incubated at 37° C., 5% CO₂, for 3 minutes. Seven milliliters DMEM complete media, with 10% FBS, 100 IU/ml of penicillin and 100 mg/ml of streptomycin sulfate (Invitrogen, Carlsbad), were added to each flask. The cell media was mixed by pipeting up and down to suspend the cells evenly. The cells were then transferred to a 50 ml Falcon (Becton Dickinson, Palo Alto) centrifuge tube. The above steps were repeated for all flasks. The cell suspensions were combined in 50 ml centrifuge tubes and centrifuged at 1200 rpm for 5 min. to pellet the cells.

The cells were washed twice in PBS as follows. The media in the tubes was discarded and the cells were resuspended in each tube using 10 ml of PBS. All cells were combined in one 50 ml Falcon centrifuge tube, and PBS was added to generate a final volume of 50 ml. The samples were centrifuged at 1200 rpm for 5 minutes. The PBS in the tubes was discarded, and the cells were resuspended in the tube using 10 ml of PBS. PBS was again added to generate a 50 ml final volume. The samples were mixed and aliquots were taken to count the cells. The samples were centrifuged at 1200 rpm for 5 minutes. The PBS in the tube was discarded and the cells were resuspended in PBS (1.0×10⁷ cells/ml) at room temperature (25° C.).

During centrifugation, 10 ml of DMEM complete media was prepared in each 15 ml Falcon centrifuge tube. The replicon RNA (1 μg) was added to a sterile microcentrifuge tube on ice. Nine (9) μg of naive Huh-7 total RNA was then added to the microfuge tube to reach a final RNA amount of 10 μg. Two micoliters of ribonuclease inhibitor (RNAsin, Promega, Madison) was added to each sample.

A Bio-Rad Gene PulserII electroporator (Bio-Rad Laboratories, Hercules, Calif.) was used for electroporation of the replicons into the Huh-7 cells, using the following general parameters: 270 V, 950 μF, and 0.4 cm Bio-Rad cuvette.

An aliquot (0.4 ml) of the Huh-7 cell suspension (see above) was added to one microcentrifuge tube, which contained an RNA sample. The sample was mixed by pipetting up and down several times. The entire RNA-cell mixture was then transferred to a 0.4 cm Bio-Rad cuvette. The electroporator was charged and then discharge pulsed. After the pulse, DMEM complete media from a 15-ml Falcon centrifuge tube (see above) was added immediately, which contained 10 ml of complete media. The mixture was transferred to the same 15-ml Falcon centrifuge tube. The sample was mixed by pipetting up and down, and the entire mixture was transferred to a 100×20 mm tissue culture dish.

The cells were incubated at 37° C., 5% CO₂, for approximately 24 hrs. The media was replaced with DMEM complete media with 500 μg/ml G418. The cells were incubated in an incubator at 37° C., 5% CO₂, for approximately 3-4 weeks until the cells were ready for picking colonies or staining. During the incubation, the selective media was replaced once a week.

E. Generation of HCV Reporter-replicon Cell Line and its Validation

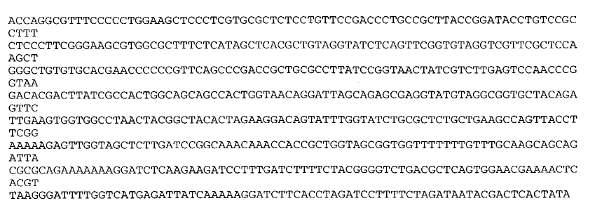

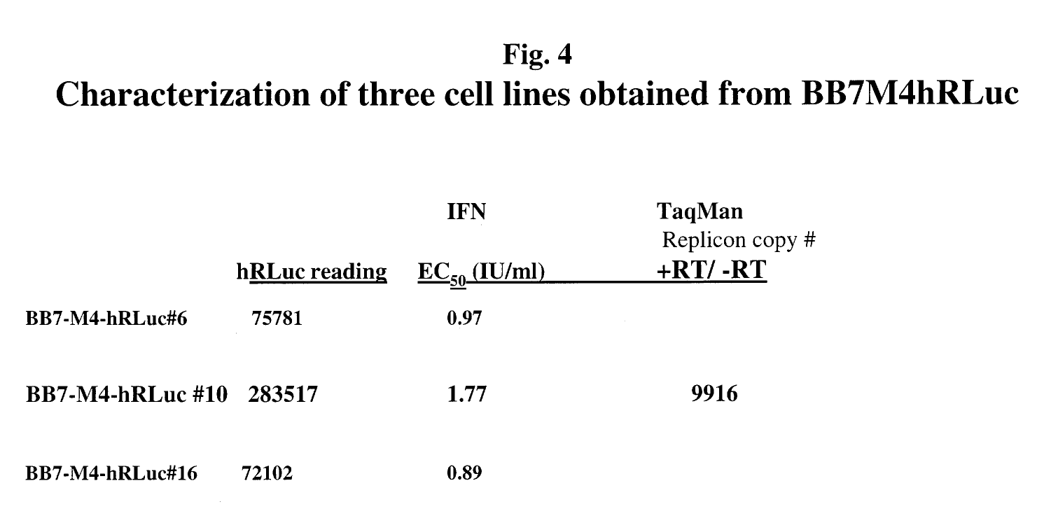

After electroporation of the BB7M4hRLuc in vitro-transcribed RNA into Huh-7 cells, 60 colonies were picked and tested for reporter gene expression. Three colonies had significant reporter gene expression above background that was stable upon expansion to a cell line (See FIG. 4). Reporter gene (hRLuc) expression was measured by seeding 2×10⁴ cells of each Huh-7 cell line containing the BB7M4hRluc replicon in a 96 well plate and incubated for 3 days at 37° C. and 5% CO₂ The media was removed by aspiration and the cells in each well were lysed with 1× passive lysis buffer (Promega, Madison, Wis.). Twenty microliters of Renilla luciferase substrate (Promega, Madison, Wis.) were added to each well and read in a Microbeta jet plate reader (Perkin Elmer, Boston, Mass.). Out of these three cell lines, #10 (BB7M4hRLuc#10) had the best expression and was chosen for validation studies. Total RNA was extracted with a QiaAmp RNA extraction kit (Qiagen, Palo Alto). Twenty microliters of the RNA were subjected to reverse transcription employing Multiscribe (ABI, Palo Alto, Calif.) according to the manufacturer's instructions and using random hexamers to generate the cDNA. For the PCR step, 10 μl of the cDNA was incubated with 1.5 μl of 10 μM of each of the primers (HCV 5UTR-127F: 5′-TCCCGGGAGAGCCATAGTG-3′; HCV 5UTR-219R:5′-GGCATTGAGCGGGTTGATC-3′), 0.5 μl of 10 μM of the probe (HCV 5UTR-168T: FAM-CCGGAATTGCCAGGACGACCG-BHQ1), 11.5 μl of distilled water and 25 μl of the 2× master mix (ABI, Palo Alto, Calif.). The incubation parameters in the ABI 7700 were: 50° C.-2 min., 95° C.-10 min. and cycle 45 times at 95° C.-15 secs and 60° C.-1 min. TaqMan RNA quantitation was done based on a standard curve run at the same time as the regular sample. In order to rule out that this cell line was not obtained due to integration of nucleic acid in the genome, the TaqMan PCR was done in the presence or absence of the reverse transcriptase.

An approximately 10,000-fold difference in signal was obtained in the presence or absence of Multiscribe (FIG. 4). This indicates that the signal obtained after amplification of the cDNA from the replicon RNA was much higher than the signal from the residual DNA after extraction. This confirmed that the signal was due to HCV RNA accumulation (replication) in the cytoplasm and not due to integration of the HCV nucleic acid into the Huh-7 genome.

In order to validate the use of the hRLuc reporter signal as an authentic indicator of HCV replication, an experiment was set up such that BB7M4hRLuc#10 cell line was incubated with four different concentrations of IFN of 30, 10, 3 and 1 IU/ml. This resulted in a dose dependent decrease in hRLuc signal with an extrapolated EC₅₀ of 1.77 IU/ml (FIG. 4). This is similar if not more sensitive to what was reported in the art. (Bartenschalger and Lohmann, Antiviral Res. 52:1-17 (2001)).

The recombinant host cells containing the nucleic acid molecules of the invention have a variety of uses. For example, the cells are useful for studying the role of nonstructural proteins and RNA elements in replication, the role of RNA elements in translation, the interaction of viral protein and RNA elements with host factors and study viral gene and RNA replication regulation, in general. The cell line BB7M4hRLuc#10 and others derived from BB7M4hRLuc can easily be adapted for screening assays for drug discovery. Preferred cells for expression purposes will be mammalian cells. Other cell types, such as bacterial, yeast, fungal, insect, nematode, and plant cells, are also possible. Examples of suitable mammalian recombinant host cells include Huh-7, VERO, HeLa, CHO, COS, BHK, HepG2, 3T3, or other mammalian cell lines.

E. Comparison of Huh-7 Cell Line BB7M4hRLuc#10 with other Available Reporter-selectable Replicon Cell Lines

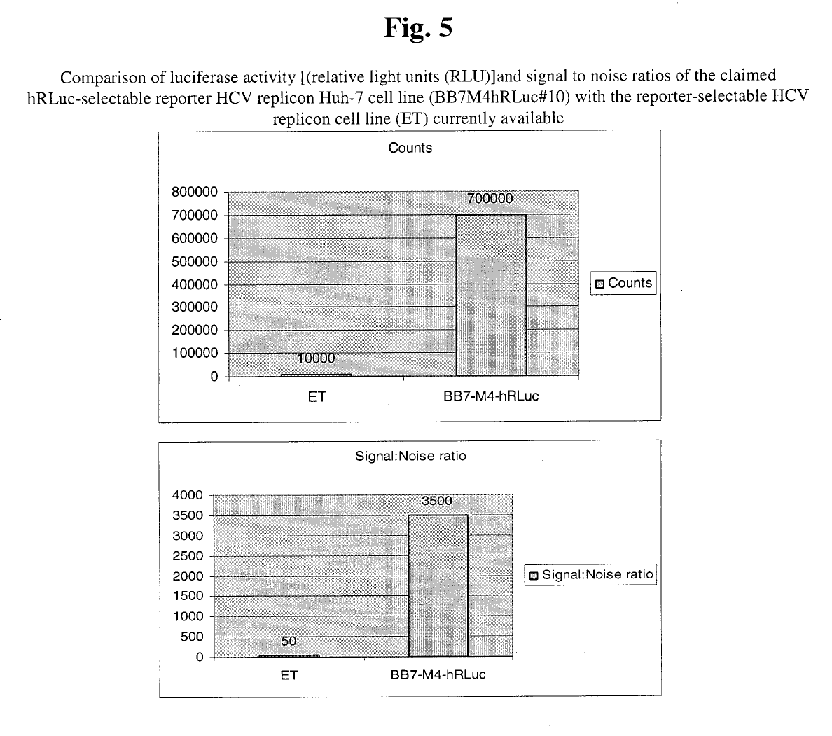

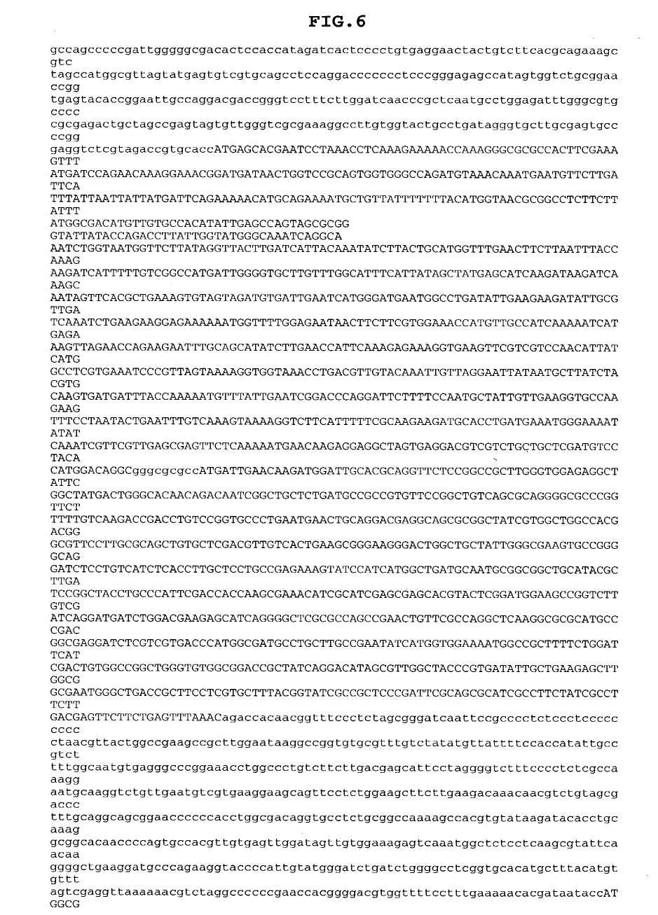

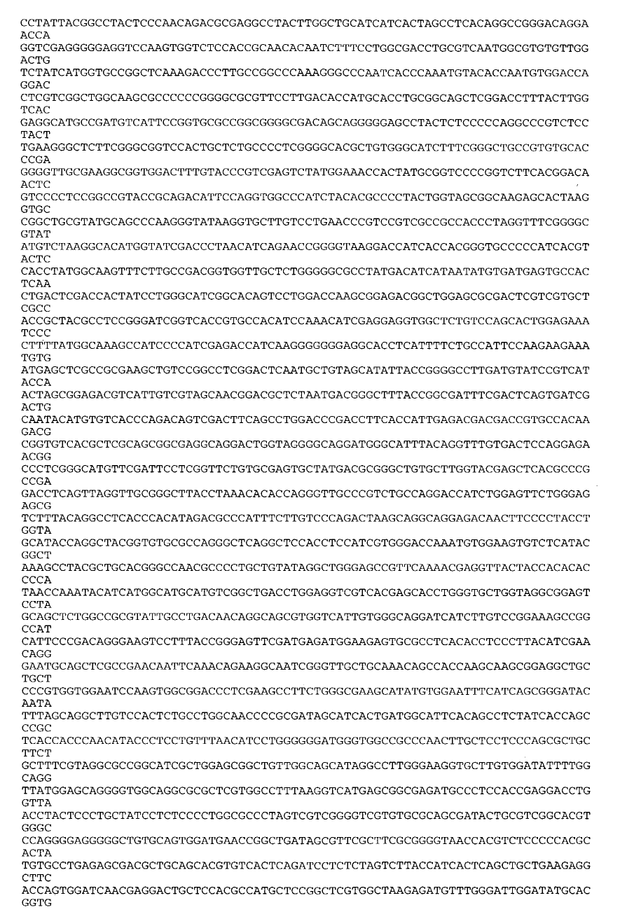

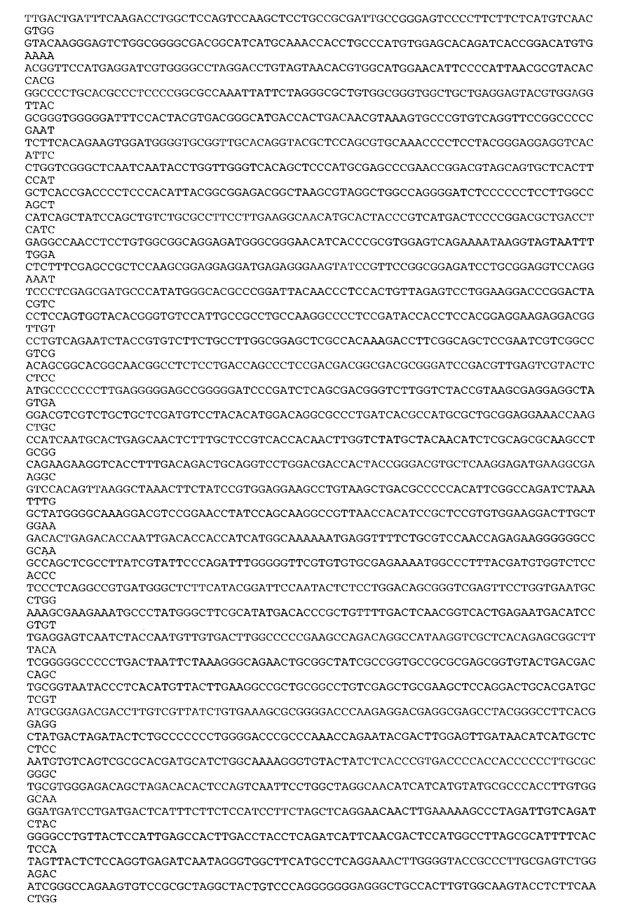

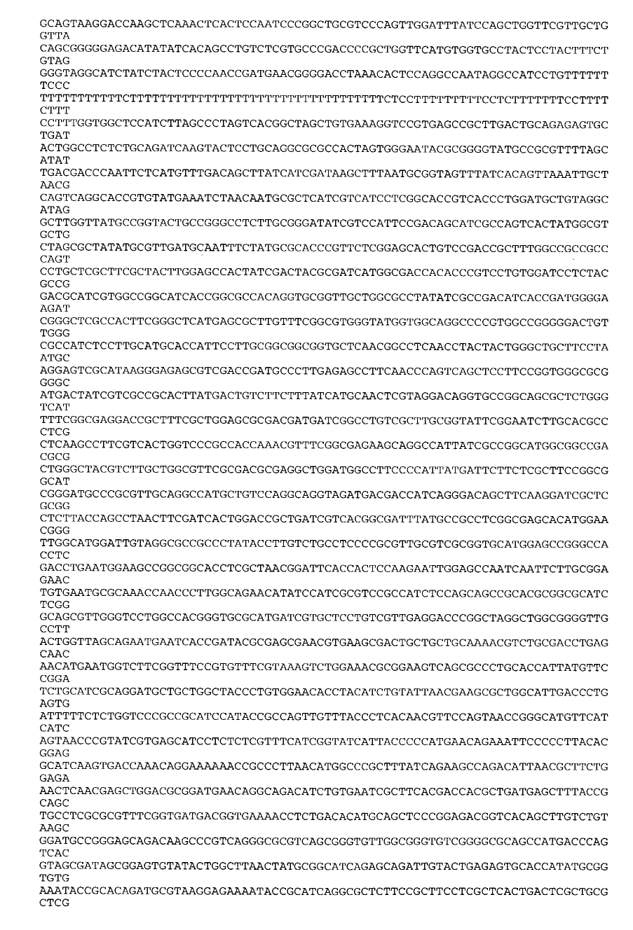

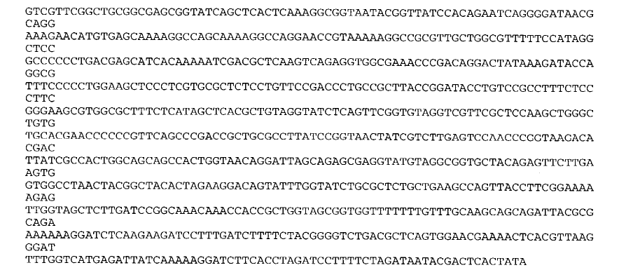

When the actual reporter (hRLuc) signal of the cell line generated from the current invention (BB7M4hRLuc#10) was compared with the signal from the other available reporter-selectable replicon cell line (ET), a significant and surprising difference was observed. The BB7M4hRLuc#10 line was capable of generating 700,000 relative light units (RLU, units for expressing luciferase activity) of reporter gene activity. Under the same conditions the ET line produced 10,000 counts of reporter gene activity (see FIG. 5). This resulted in a signal to noise ratio of 3500 for BB7M4hRLuc#10 line and 50 for the ET line, amounting to a 70 fold difference in signal to noise ratios.

While the invention has been described in terms of various preferred embodiments and specific examples, the invention should be understood as not being limited by the foregoing detailed description, but as being defined by the appended claims and their equivalents.

What is claimed is:

1. An Huh-7 cell line stably transformed with a nucleic acid molecule comprising:
(i) an HCV 5′ NTR fused to the N-terminus of a capsid coding region;
(ii) a humanized Renilla luciferase (hRLuc) nucleic acid molecule encoding a functional Renilla luciferase polypeptide, wherein said humanized Renilla luciferase nucleic acid molecule is fused at its N-terminus to said capsid and at its C-terminus to a foot and mouth disease virus (FMDV) 2A proteinase, which is fused to a neomycin phosphotransferase (NPTII) gene, wherein the hRLuc2A-NPTII fusion, upon expression from the HCV replicon, is cleaved at the 2A peptide to generate the hRLuc protein and the subsequent hRLuc signal and the NPTII protein that confers resistance to G418; and
(iii) an internal ribosome entry site (IRES) from encephalomyocarditis virus (EMCV), inserted downstream of the NPTII gene, which directs translation of HCV proteins NS3 to NS5B; and
(iv) an HCV NS3-5B polyprotein coding region containing adaptive mutations.

2. The cell line according to claim 1, wherein the adaptive mutations in the HCV NS3-5B polyprotein-coding region are E1202G, T1208I and S2197P.

3. The cell line according to claim 2, wherein the nucleic acid molecule is a self-replicating RNA molecule of BB7M4hRLuc.

4. The cell line according to claim 3, wherein the nucleic acid molecule comprises SEQ ID: 1.

5. An HCV double-stranded cDNA, which can be transcribed in vitro to produce replicating HCV RNA transcripts, wherein the cDNA comprises:
(i) an HCV 5′ NTR fused to the N-terminus of a capsid coding region;
(ii) a humanized Renilla luciferase (hRLuc) nucleic acid molecule encoding a functional Renilla luciferase polypeptide, wherein said humanized Renilla luciferase nucleic acid molecule is fused at its N-terminus to said capsid and at its C-terminus to a foot and mouth disease virus (FMDV) 2A proteinase, which is fused to a neomycin phosphotransferase (NPTII) gene, wherein upon translation the hRLuc2A-NPTII fusion is released by a self-cleaving peptide;
(iii) an internal ribosome entry site (IRES) from an encephalomyocarditis virus (EMCV), inserted downstream of the NPT II gene, which directs translation of HCV proteins NS3 to NS5B; and
(iv) an HCV NS3-5B polyprotein coding region containing adaptive mutations.

6. An HCV double-stranded cDNA according to claim 5, wherein the hRLuc2A-NPTII fusion upon expression is cleaved at the 2A peptide to generate the hRLuc protein and the subsequent hRLuc signal and the NPTII protein that confers resistance to G418.

7. A HCV double-stranded cDNA according to claim 6, wherein the adaptive mutations in the HCV NS3-5B polyprotein-coding region are E1202G, T1208I and S2197P.

8. A cell containing the cDNA according to claim 7.

9. A cell containing the cDNA according to claim 8, wherein the cell is a prokaryote.

10. An HCV double-stranded cDNA according to claim 7, wherein translation of the fusion protein encoding hRLuc protein, FMDV 2A peptide and the NPTII protein of said nucleic acid molecule results in the separation of the hRLuc and NPTII proteins during protein synthesis by self-cleavage by the FMDV 2A peptide.

11. A method of generating an Huh-7 cell line stably replicating an HCV hRLuc-selectable subgenomic replicon RNA, comprising the steps of:
(a) constructing a nucleic acid molecule comprising RNA sequences encoding the HCV replicon, wherein said nucleic acid molecule comprises
(i) an HCV 5′ NTR fused to a capsid coding region.
(ii) a humanized Renilla luciferase (hRLuc) nucleic acid molecule encoding a functional Renilla luciferase polypeptide, wherein said humanized Renilla luciferase nucleic acid molecule is fused at its N-terminus said capsid and at its C-terminus to a foot and mouth disease virus (FMDV) 2A proteinase, which is fused to a neomycin phosphotransferase (NPTII) gene, wherein the hRLuc2ANPTII fusion upon expression is cleaved at the 2A peptide to generate the hRLuc protein and the subsequent hRLuc signal and the NPTII protein that confers resistance to G418;
(iii) an internal ribosome entry site (IRES) from an encephalomyocarditis virus (EMCV), inserted downstream of the NPT II gene, which directs translation of HCV proteins NS3 to NS5B;
(iv) an HCV NS3-5B polyprotein coding region containing adaptive mutations; and
(b) stably transforming Huh-7 cells with the nucleic acid molecule from step (a).

12. A method according to claim 11, wherein the adaptive mutations in the HCV NS3-5B polyprotein-coding region are E1202G, T1208I and S2197P.

13. An assay using a mammalian cell line derived by transfection of a reporter-selectable HCV replicon according to claim 10, wherein the assay is used to determine the specific antiviral activity of inhibitors in standard dose responses by using reporter gene activity as an endpoint.

14. An assay according to claim 13, wherein the reporter gene activity is from hRLuc.

15. An assay according to claim 14, wherein the cell line is Huh-7.

16. An assay according to claim 15, wherein the reporter gene activity is from hRLuc.


SEQUENCE LISTING (13 sequences)

SEQ ID NO: 1 - DNA, 12315 bp, Unknown
  A cell line wherein the nucleic acid molecule is a self replicating RNA molecule.
ctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggagccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagcagctgttgaattttgaccttcttaagcttgcgggagacgtcgagtccaaccctgggcccgggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccccccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggacctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactcctgcaggcgcgccactagtgggaatacgcggggtatgccgcgttttagcatattgacgacccaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttctagataatacgactcactata

SEQ ID NO: 2 - DNA, 12305 bp, Unknown
  A cell line wherein the nucleic acid molecule is a self replicating RNA molecule.
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcgggtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaagaggaggctagtgaggacgtcgtctgctgctcgatgtcctacacatggacaggcgggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctatccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggacctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactcctgcaggcgcgccactagtgggaatacgcggggtatgccgcgttttagcatattgacgacccaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttctagataatacgactcactata

SEQ ID NO: 3 - DNA, 32 bp, Unknown
  A primer used for making amino acid changes.
gccagctcatcagctagccagctgtctgcgcc

SEQ ID NO: 4 - DNA, 31 bp, Unknown
  A primer used for making amino acid changes.
gcgcagacagctggctagctgatgagctggc

SEQ ID NO: 5 - DNA, 55 bp, Unknown
  A primer used for making amino acid changes.
ggactttgtacccgtcgagtctatgggaaccactatgcggtccccggtcttcacg

SEQ ID NO: 6 - DNA, 55 bp, Unknown
  A primer used for making amino acid changes.
cgtgaagaccggggaccgcatagtggttcccatagactcgacgggtacaaagtcc

SEQ ID NO: 7 - DNA, 57 bp, Unknown
  A primer used for making amino acid changes.
ggcacatggtatcgaccctaacatcagaatcggggtaaggaccatcaccacgggtgc

SEQ ID NO: 8 - DNA, 57 bp, Unknown
  A primer used for making amino acid changes.
gcacccgtggtgatggtccttaccccgattctgatgttagggtcgataccatgtgcc

SEQ ID NO: 9 - DNA, 56 bp, Unknown
  A primer used for making amino acid changes.
gcgtaggctggccaggggatctcccccccccttggccagctcatcagctagccagc

SEQ ID NO: 10 - DNA, 56 bp, Unknown
  A primer used for making amino acid changes.
gctggctagctgatgagctggccaagggggggggagatcccctggccagcctacgc

SEQ ID NO: 11 - DNA, 39 bp, Unknown
  A primer used for making amino acid changes.
ccaggcgcgccatggcttccaaggtgtacgaccccgagc

SEQ ID NO: 12 - DNA, 75 bp, Unknown
  A primer used for making amino acid changes.
gactcgacgtctcccgcaagcttaagaaggtcaaaattcaacagctgctgctcgttcttcagcacgcgctccacg

SEQ ID NO: 13 - DNA, 64 bp, Unknown
  A primer used for making amino acid changes.
ccaggcgcgcccgggcccagggttggactcgacgtctcccgcaagcttaagaaggtcaaaattc